(19) United States
(12) Patent Application Publication
(10) Pub. No.: US 2025/0127990 A1
(43) Pub. Date: Apr. 24, 2025

(54) Medical Injection Device and Method for Assembling This Injection Device

(71) Applicants: Becton, Dickinson and Company, Franklin Lakes, NJ (US); Becton Dickinson France, Le Pont-de-Claix (FR)

(72) Inventors: Nicolas Euvrard, London (GB); Alfred Leibbrand, Claix (FR)

(21) Appl. No.: 18/696,048

(22) PCT Filed: Sep. 30, 2022
(86) PCT No.: PCT/EP2022/077287

§ 371 Date: Mar. 27, 2024

(30) Foreign Application Priority Data
Oct. 1, 2021 (EP) 21306378.7

(51) Int. Cl. A61M 5/31

(52) U.S. Cl. CPC A61M 5/3134; A61M 2205/60; A61M 2207/10

(57) ABSTRACT

This injection device includes a medical container provided with a tubular barrel extending along a longitudinal axis (A) and defining a reservoir for containing a medical product, said tubular barrel having a distal shoulder and a proximal end. A distal tip longitudinally protrudes from the distal shoulder of said tubular barrel, the distal tip defining an axial passageway in fluid communication with the reservoir so that the medical product is expelled through said distal tip. An adaptor is secured to the the distal tip, the adaptor being configured to allow connection of a connector to the distal tip. A supporting ring includes a Radio Frequency Identification, RFID, tag configured to permit individual identification of the injection device. The RFID tag includes an RFID chip and an RFID antenna, while said supporting ring has an opening allowing mounting of the supporting ring around the distal tip such that the RFID tag is located between a proximal end of the adaptor and the distal shoulder of the barrel.

### CROSS-REFERENCE TO RELATED APPLICATIONS

This application is the United States national phase of International Application No. PCT/EP2022/077287 filed Sep. 30, 2022, and claims priority to European Patent Application No. 21306378.7 filed Oct. 1, 2021, the disclosures of which are hereby incorporated by reference in their entireties.

### BACKGROUND OF THE INVENTION

### Field of the Invention

The present invention relates to a medical injection device, and a method for assembling this injection device.

In this application, the distal end of a component or of a device is to be understood as meaning the end furthest from the user's hand and the proximal end is to be understood as meaning the end closest to the user's hand. Likewise, in this application, the “distal direction” is to be understood as meaning the direction of injection, with respect to a medical container of the invention, and the “proximal direction” is to be understood as meaning the opposite direction to said direction of injection, that is to say the direction towards the user's hand holding a container as for an injection operation.

### Description of Related Art

Medical injection devices, such as pre-fillable or prefilled syringes, usually comprise a hollow body or barrel forming a container for a medical product. This tubular body comprises an opened proximal end provided with a plunger rod for pushing a stopper arranged inside the barrel, a flange around said opened proximal end for allowing a user to place his or her fingers, and a distal end in the form of a longitudinal tip defining an axial passageway through which the medical product is expelled from the container. However, this longitudinal tip does not allow parenteral administration by itself and must either comprise a staked needle or an adaptor allowing the connection of the syringe to a connector such as a needle hub or an intravenous (IV) line. Basically, adaptors may be secured around the longitudinal tip of the syringe by gluing, screwing, snap-fitting or friction force. The connector is then mounted on the adaptor, for example by screwing.

There is an increasing need for individual traceability of the injection devices from the manufacturing process until the final labeling, the final use or the disposal of said injection devices.

It is known for example from document WO2017157784 a receptacle having a cylindrical lateral surface surrounded by a sequence of printed machine-readable unique identifier codes. These printed unique identifier codes allow tracking and tracing of each receptacle along a supply chain. However, these unique identifier codes are printed on an external side of the receptacle so that they may be removed or damaged for example during handling or use of the receptacle. Moreover, the unique identifier codes cover a portion of the receptacle so that they may have an impact on a customer visual inspection process.

The document U.S. Pat. No. 20,210,236736 discloses a tamper evident assembly with RFID for syringes. The document WO2020260298 discloses an adaptor for a medical container and an RFID tag at least partially embedded into said adaptor.

### SUMMARY OF THE INVENTION

In this context, an object of the present invention is to provide a medical injection device that alleviates the above-mentioned drawbacks by allowing individual identification of a medical injection device with low or no impact on visual inspection, with few or no risks of being removed or damaged, and with a limited impact on the manufacturing process.

An aspect of the invention is an injection device comprising a medical container provided with:
a tubular barrel extending along a longitudinal axis A and defining a reservoir for containing a medical product, said tubular barrel having a distal shoulder and a proximal end,
a distal tip longitudinally protruding from the distal shoulder of said tubular barrel, the distal tip defining an axial passageway in fluid communication with the reservoir so that the medical product is expelled through said distal tip,
an adaptor secured to the the distal tip, the adaptor being configured to allow connection of a connector to the distal tip, and
a supporting ring including a Radio Frequency Identification, RFID, tag configured to permit individual identification of the injection device, wherein the RFID tag includes a RFID chip and a RFID antenna, said supporting ring having an opening allowing mounting of the supporting ring around the distal tip such that the RFID tag is located between a proximal end of the adaptor and the distal shoulder of the barrel.

The medical injection device of the invention thus allows identification and tracking of the medical container with no impact on visual inspection since the RFID tag does not hinder inspection of the tubular barrel and the medical product contained therein.

The risks of removal or damage to the RFID tag are removed or limited since the RFID tag is blocked between the adaptor and the tubular barrel. Unlike solutions where the tag is embedded in a removable part of the medical device, where the RFID tag is located on a part grasped by a user, such as a syringe tip cap, or where the RFID tag is attached to the medical device via an adhesive, the RFID tag of the injection device of the invention remains attached to the device during and after the medication has been delivered.

Advantageously, the opening of the supporting ring is a through opening located at the center of said supporting ring.

The impact on the manufacturing process is also limited since the supporting ring including the RFID tag may be easily mounted on the distal tip by insertion of the distal tip through the central opening of the supporting ring. Compared to solutions consisting in over-molding an RFID tag into a plastic component, the medical injection device according to the invention enables to leverage on an existing manufacturing method (i.e. PRTC assembly) without need to change the molds.

The supporting ring is preferably a disc-shaped ring, and the opening is preferably a circular through opening.

In an embodiment, the supporting ring has an inner diameter that is lower than an outer diameter of the distal tip.

The inner diameter of the supporting ring may also refer to the maximal diameter of the opening of the supporting ring.

Therefore, the RFID tag is secured to the distal tip by interference fit.

In an embodiment, the supporting ring has an outer diameter that is equal to or lower than an outer diameter of the adaptor, and/or of a tip cap when the injection device includes a tip cap connected to the adaptor for sealing the axial passageway of the distal tip.

This enables to limit the radial dimension of the injection device so that no change of packaging is required. This also limits the risks that the RFID tag be damaged, since the RFID tag does not protrude from the adaptor in a radial direction.

In an embodiment, the supporting ring defines a distal abutment face configured to abut against the proximal end of the adaptor.

Preferably, the distal abutment face is flat.

This permits load distribution when pushing the RFID tag in the proximal direction by means of the proximal end of the adaptor abutting against the supporting ring. Accordingly, the risk that the RFID tag is deformed and damaged is limited.

In one embodiment, when mounted on injection device, the supporting ring has a flat distal face and a flat opposite proximal face, and extends in a plane orthogonal to the longitudinal axis A.

In an embodiment, the supporting ring is secured to a proximal-most portion of the distal tip.

The proximal-most portion of the distal tip connects the distal tip to the distal shoulder of the barrel. This proximal-most portion may thus have a proximally flaring diameter. The outer surface of the proximal-most portion has a concave shape.

In an embodiment, the supporting ring has a bevel extending around the opening, on a proximal face thereof.

This permits to position the RFID tag as close as possible to the distal shoulder of the barrel, such that there is no need to increase the distal tip length in order to mount the supporting ring thereon.

The bevel of the supporting ring and the proximal-most portion of the distal tip may have a complementary shape.

In an embodiment, the supporting ring has inward radial legs defining the opening and configured to secure the supporting ring to the distal tip.

The legs allow mounting of the RFID tag around the distal tip with limited risks that the RFID tag be deformed and thus damaged during assembly of the injection device. The legs limit internal stress in the RFID tag. Without legs, more material of the supporting ring has to deform, which leads to higher axial forces exerted on said supporting ring during assembly and consequently higher internal stress in the RFID tag, thus potentially damaging the tag.

Preferably, the distal tip comprises a groove configured to receive an inner ring of the adaptor, and said groove length is comprised between 1 mm and 2 mm, and is preferably about 1.5 mm. Preferably, when mounted on the injection device, the supporting ring comprising the RFID tag is also received within the groove of the distal tip. This groove may be cylindrical, so as to stabilize the supporting ring. That is, there is no slope that tend to move the supporting ring on one side or another. Furthermore, having the supporting ring in the groove of the distal tip provides a clear indication that the supporting ring is properly positioned. During assembly, this indication may be a drop in the axial force that pushes the supporting ring downwards (in the proximal direction) and possibly a sound, such as a “click”.

In an embodiment, the RFID antenna and the RFID chip are embedded, preferably overmolded, in the supporting ring.

The RFID antenna and chip are accordingly protected from the outside environment.

In an embodiment, the supporting ring is a Printed Circuit Board, PCB, the RFID antenna is directly printed on said PCB, preferably on a distal face thereof, and the RFID chip is welded to the RFID antenna.

This limits the axial dimension of the supporting ring and thus limits the length of the distal tip, thereby limiting risks of breakage of this distal tip.

Preferably, the antenna is in the form of a single loop, a folded loop or a cut loop.

By single loop it is meant that the antenna has its two ends connected to the chip, so as to form a closed loop. By folded loop it is meant that the antenna includes two symmetrical branches each having one end connected to the chip and a free end arranged such that each branch forms at least one fold between these two ends. By cut loop it is meant that the antenna has two symmetrical branches each having one end connected to the chip and a free end, without forming any fold between these two ends. In the folded loop and cut loop configuration, the two branches of the antenna may extend in a different half of the supporting ring with regard to a longitudinal plane including the longitudinal axis A.

In an embodiment, the RFID tag is a Low Frequency Radio Frequency Identification, LF-RFID, tag, a High Frequency Radio Frequency Identification, HF-RFID, tag or, preferably, a Ultra High Frequency Radio Frequency Identification, UHF-RFID, tag.

The UHF-RFID tags permit to increase the reading range and thus ease the identification of the injection device.

Another aspect of the invention is a method for assembling the above-described injection device, said method comprising the step of mounting the supporting ring onto the distal tip before mounting the adaptor onto said distal tip, such that the supporting ring including the RFID tag is arranged between a proximal end of the adaptor and a distal end of the barrel.

In one embodiment, the method comprises the steps of:
(i) inserting the opening of the supporting ring onto the distal tip;
(ii) moving the supporting ring in the proximal direction relative to the distal tip, until an inner diameter of the supporting ring interferes with an outer diameter of the distal tip;
(iii) pushing the supporting ring in the proximal direction in order to move the supporting ring into a groove of the distal tip;
(iv) securing the adaptor to the distal tip.

In an embodiment, the proximal movement of the supporting ring relative to the distal tip in step (ii) is accomplished by gravity.

That is, the distal tip is in a vertical position and the supporting ring supporting the RFID tag moves downwards due to gravity.

In an embodiment, step (iii) includes the supporting ring being pushed further in the proximal direction by the proximal end of the adaptor abutting against the supporting ring, until the adaptor reaches its final position relative to the distal tip.

The final position is reached when the inner ring of the adaptor is secured into the groove of the distal tip.

### BRIEF DESCRIPTION OF THE DRAWINGS

The terms Fig., Figs., Figure, and Figures are used interchangeably in the specification to refer to the corresponding figures in the drawings.

The invention and the advantages arising therefrom will clearly emerge from the detailed description that is given below with reference to the appended drawings as follows:

FIG. 1 is a perspective view of a medical injection device according to an embodiment of the invention,

FIGS. 2A and 2B are cross-section views of a medical injection device according to an embodiment of the invention,

FIG. 3A is a perspective view of a RFID tag of a medical injection device according to an embodiment of the invention,

FIG. 3B is a bottom view of a proximal face of a RFID tag of a medical injection device according to an embodiment of the invention,

FIG. 4 is a perspective view of a RFID tag of a medical injection device according to an embodiment of the invention,

FIGS. 5A to 5C are perspective and partially cut views of a disc-shaped ring of a medical injection device according to an embodiment of the invention,

FIGS. 6A-6E are cross-section views illustrating different steps for assembling a medical injection device according to an embodiment of the invention.

### DESCRIPTION OF THE INVENTION

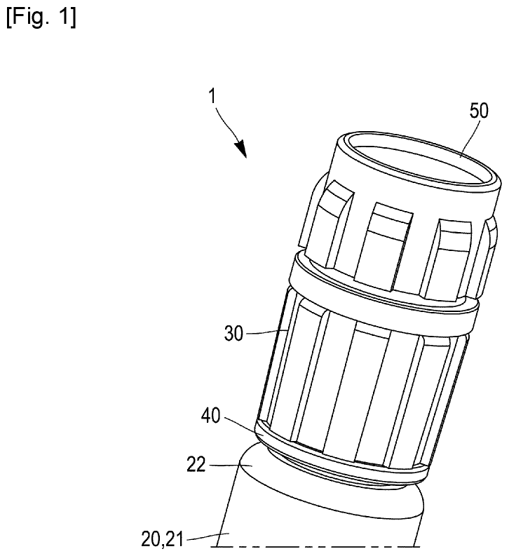

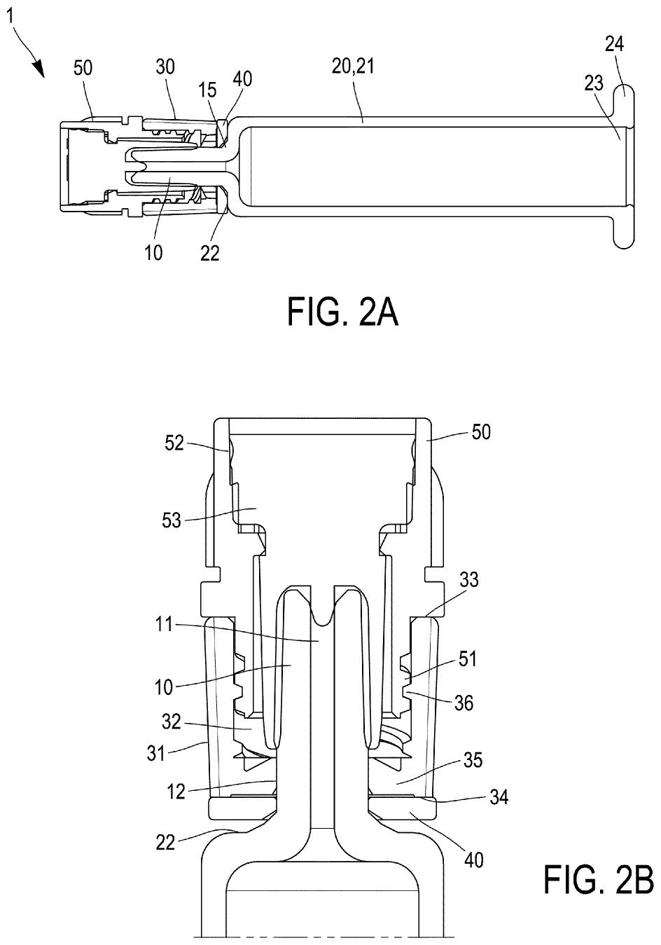

With reference to FIGS. 1, 2A and 2B is shown an injection device 1 according to an embodiment of the invention. The medical injection device 1 comprises a medical container 20, such as a prefillable or prefilled syringe, an adaptor 30 for allowing connection of the medical container 20 to a connector, such as a needle hub (not shown) or an IV line (not shown), and a supporting ring such as a disc-shaped ring 40 including a Radio Frequency Identification (RFID) tag 60. Before use, for example during storage or transport, the injection device 1 may include a tip cap assembly 50.

The medical container 20 comprises a tubular barrel 21 defining a reservoir for containing a medical product. The tubular barrel 21 may be made of a plastic or a glass material. With reference to FIGS. 2A and 2B, the barrel 21 has a distal end or shoulder 22 provided with a distal tip 10 longitudinally extending along a longitudinal axis A, and an opposite opened proximal end 23 which may be provided with a finger flange 24. The opened proximal end 23 may receive a plunger rod (not shown) for pushing a stopper (not shown) arranged inside the tubular barrel 21 in order to expel a medical product contained in the tubular barrel 21.

The distal tip 10 distally protrudes from the distal end 22 of the tubular barrel 21. The distal tip 10 has an axial passageway 11 in fluid communication with the reservoir so that the medical product is expelled through the passageway 11. The distal tip 10 is devoid of a needle and therefore needs to receive an adaptor 30 allowing connection of the distal tip 10 with a connector for delivering the medical product to a patient. As above mentioned, the connector may be a needle hub or an IV line. The distal tip 10 has a proximal portion configured to receive the adaptor 30, this proximal portion may include a groove 12 and/or ribs 13, 14 to secure the adaptor 30 thereon by snap-fitting, interference fitting or gluing. Typically, the distal tip 10 comprises two ribs 13, 14 in between is located the groove 12. The distal tip 10 also includes a proximal-most portion 15 having a proximally increasing diameter, said proximal-most portion 15 connecting the distal tip 10 to a distal shoulder 22 of the barrel 21.

The adaptor 30 is configured to allow the connection of a connector to the distal tip 10 of the medical container 20 and to secure said connector to the distal tip 10. To that end, the adaptor 30 includes a tubular body 31 defining a distal end 33, a proximal end 34, and an inner cavity 32 for receiving the connector, this inner cavity 32 comprising a connecting element, such as an inner thread 36, configured to engage a corresponding connecting feature of the connector, such as an outer thread or outer wings of a needle hub. Before use, this inner thread 36 may engage an outer thread 51 of the tip cap assembly 50 as shown in FIG. 2B.

The adaptor 30 further includes an inner ring 35 inwardly protruding form an inner wall of the tubular body 31. The inner ring 35 is configured to engage the proximal portion of the distal tip 10 so as to secure the adaptor 30 to the distal tip 10 for instance by gluing, snap-fitting or friction force. For example, the inner ring 35 may have resilient legs that engage the groove 12 of the distal tip 10.

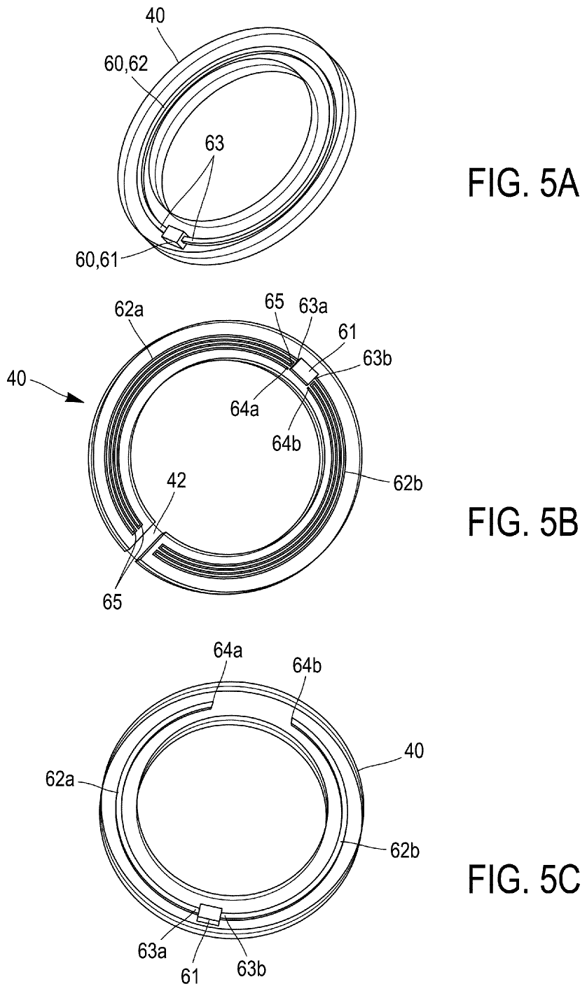

The RFID tag 60 is configured to allow individual identification and tracking of the injection device 1. To that end, as shown in FIG. 5A-5C, the RFID tag 60 includes a RFID chip 61, which has a storing unit containing a Unique Device Identifier (UDI), and an RFID antenna 62. The RFID chip 61 and antenna 62 are supported by a support, said support being in the form of a supporting ring 40, such as a disc-shaped ring.

The supporting ring 40 is mounted around the distal tip 10, on the proximal portion or preferably on the proximal-most portion 15 of the distal tip 10, and is located between a proximal end 34 of the adaptor 30 and a distal shoulder 22 of the tubular barrel 21 so that removal of the supporting ring 40 is prevented during transport, storage or use of the injection device 1.

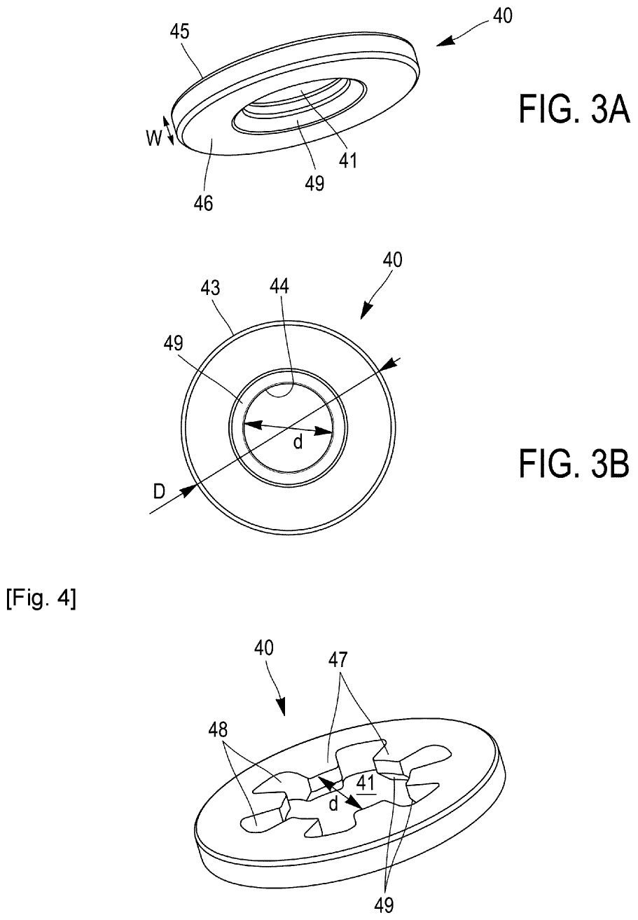

As visible in FIG. 3A, the disc-shaped supporting ring 40 defines a central through-opening 41 allowing insertion of the distal tip 10. The disc-shaped supporting ring 40 may be a fully closed ring (FIGS. 5A, 5C) or a split ring (FIG. 5B). If a split ring, the RFID chip 61 is preferably arranged diametrically opposite a slot 42. The RFID antenna 62 may thus be symmetrical with regard to a longitudinal plane including the longitudinal axis A, in order to maximize performance.

The supporting ring 40 may have circular outer and inner edges 43, 44. With reference for example to FIG. 3B, the inner edge 44 defines an inner diameter d that may be lower than an outer diameter of the distal tip 10 so that the RFID tag 60 is secured to the distal tip 10 preferably by interference fit. When the disc-shaped ring 40 is in the groove 12 of the distal tip 10, the inner diameter d may be between 0.05 mm and 0.4 mm lower than the outer diameter of the distal tip 10. When the disc-shaped ring 40 is above the groove 12, where the interference is maximum, the inner diameter d may be between 0.1 mm and 0.5 mm lower than the outer diameter of the distal tip 10.

It is also noted that the inner edge 44 of the supporting ring 40 may include a bevel 49 located on the proximal side, this bevel 49 easing the mounting of the supporting ring 40 onto the distal tip 10. The bevel 49 also permits to position the supporting ring 40 as far as possible in the proximal direction, i.e. as close as possible to the barrel 21, so that the supporting ring 40 does not take too much of the distal tip 10 axial dimension. In order to further reduce the axial dimension taken by the supporting ring 40 along the distal tip 10, the disc-shaped ring 40 is flat, i.e. includes a distal face 45 and an opposite proximal face 46 which are preferably orthogonal to the longitudinal axis A. The axial dimension w of the supporting ring 40 may be comprised between 0.5 mm and 1.5 mm.

It is contemplated that the distal face 45 of the disc-shaped support also serves as an abutment surface configured to abut against the proximal end 34 of the adaptor 30 in order the supporting ring 40 to be pushed in the proximal direction by said adaptor 30 during assembly of the injection device 1. Having a flat distal face 45 also allows load distribution on the disc-shaped ring 40, thereby reducing the risks that the RDIF tag 60 be damaged during assembly.

As illustrated in FIG. 4, in order to ease insertion of the distal tip 10 through the central opening 41, the disc-shaped ring 40 may have inwardly extending legs 47 configured to slightly deform while the RFID tag 60 slides against the distal tip 10 during assembly of the injection device 1. The legs 47 are regularly distributed in a circumferential direction and may be separated by radial cavities 48. These legs 47 also limit deformation of the RFID chip 61 and antenna 62 during assembly. Thus, risks of damages are limited.

With reference to FIG. 3B, the disc-shaped ring 40 has an outer diameter D that is equal to or lower than an outer diameter of the tip cap assembly 50 (if any), or of the barrel 21, or preferably of the adaptor 30, more specifically of the proximal end 34 of the adaptor 30 as visible in FIG. 2B. Preferably, the outer diameter D of the supporting ring 40 is as close as possible to the outer diameter of the adaptor 30.

The RFID tag 60 may be a Low Frequency (about 30-300 kHz, preferably 125-135 kHz, and more specifically 124 kHz, 125 kHz, or 134 kHz) RFID (LF-RFID) tag, preferably a High Frequency (about 3-30 MHz) RFID (HF-RFID) tag or, more preferably, an Ultra High Frequency (about 400-1000 MHz) RFID (UHF-RFID) tag. A RFID reader can for example read the LF-RFID tag at a distance up to about 10 cm, the HF-RFID tag at a distance of about one meter and the UHF-RFID tag at a distance of about twelve meters. In one embodiment, the RFID tag 60 may be a High-Frequency Near Field Communication (HF-NFC) tag. The frequency is usually about 13.56 MHz. In this embodiment, a NFC reader can for example read the HF-NFC tag at a distance up to a few centimeters. HF-NFC differs from HF-RFID in that it can be read by a NFC smartphone. The RFID tag 60 may be even be a Bluetooth tag.

With reference to FIGS. 5A to 5C, the antenna 62 may be in the form of a single loop (FIG. 5A), a folded loop (FIG. 5B) or a cut loop (FIG. 5C). The single loop antenna 62 has two ends 63 connected, for instance welded, to the chip 61, and thus forms a closed loop. The folded loop antenna 62 includes two symmetrical branches 62a, 62b. Each of these branches 62a, 62b has one end 63a, 63b connected, for instance welded, to the chip 61 and a free end 64a, 64b arranged such that the branch 62a, 62b forms at least one fold 65 between these two ends. The cut loop antenna 62 has two symmetrical branches 62a, 62b, each of these branches 62a, 62b having one end 63a, 63b connected, for instance welded, to the chip 61 and a free end 64a, 64b. The branches 62a, 62b do not form any fold 65 between these two ends. In the folded loop antenna 62 and the cut loop antenna 62, the two branches 62a, 62b extend in a different half of the disc-shaped ring 40 with regard to a longitudinal plane including the longitudinal axis A. In the folded loop and cut loop antenna 62, the supporting ring 40 40 may be a split ring and the chip 61 and the slot 42 that splits the ring 40 are preferably diametrically opposite.

In one embodiment, the support formed by the disc-shaped ring 40 encapsulates the RFID chip 61 and antenna 62 so as to protect them from the outside environment. For instance the RFID chip 61 and antenna 62 are overmolded with the disc-shaped support. In another embodiment, the support formed by the disc-shaped ring 40 is a printed circuit board (PCB), the RFID antenna 62 being directly printed on this PCB, preferably on the distal face thereof, and the RFID chip 61 being welded to the antenna 62.

The RFID tag 60 may include a wet inlay, dry inlay, or pressure sensitive label affixed to the disc-shaped ring 40.

It is noted that the disc-shaped ring 40 is preferably made in a rigid material, such as for instance polypropylene (PP), polyethylene (PE), polyethylene terephthalate (PET), polystyrene (PS) or polycarbonate (PC), thereby limiting the risks of damage to the RFID chip 61 and antenna 62 when the supporting ring 40 is mounted onto the distal tip 10. Heat-resistant materials such as ceramics, nylon or polytetrafluoroethylene (PTFE) like Teflon® ca be used as well to prevent damage from high temperatures.

With reference to FIG. 2B, the tip cap assembly 50 may include an outer cap 52 defining a cavity for receiving an inner cap 53. The outer cap 52 is made of a rigid material while the inner cap 53 is made of a resilient, softer material than the outer cap 52. The inner cap 53 is intended to seal the passageway 11 of the medical container distal tip 10. The outer cap 52 has an outer thread 51 engaging the inner thread 36 of the adaptor 30 so as to secure the tip cap assembly 50 to the adaptor 30 and to the medical container 20.

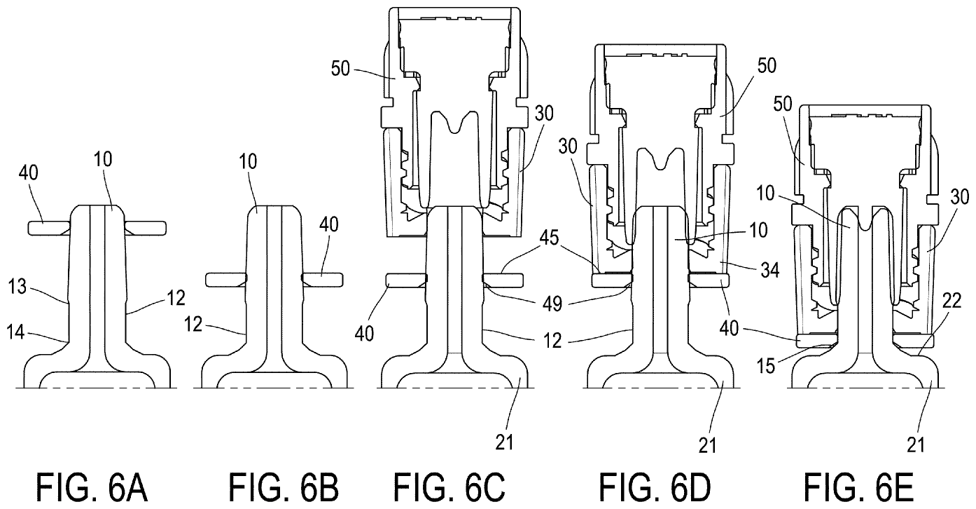

A method for assembling the injection device 1 of the invention is described below with reference to the FIGS. 6A to 6E.

The supporting ring 40 is firstly mounted onto the distal tip 10 of the injection device 1, by inserting said distal tip 10 in the opening 41 of the disc-shaped ring 40 (FIG. 6A). The disc-shaped ring 40 moves downwards, in the proximal direction, preferably due to the gravity, until its inner edge 44 interferes with the outer surface of the distal tip 10 (FIG. 6B). At this stage, the disc-shaped ring 40 may be blocked by a distal rib 13 at a distal end of the groove 12. The tip cap assembly 50 and the adaptor 30, which are already connected to each other by engagement of their threads, are then mounted onto the distal tip 10, by inserting the distal tip 10 through the inner ring 35 (FIG. 6C) of the adaptor 30. The proximal end 34 of the adaptor 30 comes in abutment against the distal face 45 of the disc-shaped ring 40 (FIG. 6D), such that the adaptor 30 pushes the disc-shaped ring 40 further in the proximal direction. The disc-shaped ring 40 may slightly deform to pass beyond the distal rib, 13 and then slides along the groove 12 and goes on moving towards the barrel 21 until the adaptor 30 reaches its final position, wherein the adaptor 30 is secured to the distal tip 10, i.e. when the inner ring 35 of the adaptor 30 properly fits within the groove 12 of the distal tip 10 (FIG. 6E). The disc-shaped ring 40, including the RFID tag 60, is now blocked between the adaptor 30 and the barrel 21, and engages the proximal-most portion 15 of the distal tip 10. Also, the disc-shaped ring 40 cannot rotate around the distal tip 10 because of the interference fit and thus remains fixed relative to the distal tip 10.

1. An injection device comprising a medical container provided with:
a tubular barrel extending along a longitudinal axis (A) and defining a reservoir for containing a medical product, said tubular barrel having a distal shoulder and a proximal end,
a distal tip longitudinally protruding from the distal shoulder of said tubular barrel, the distal tip defining an axial passageway in fluid communication with the reservoir so that the medical product is expelled through said distal tip,
an adaptor secured to the the distal tip, the adaptor being configured to allow connection of a connector to the distal tip, and
a supporting ring including a Radio Frequency Identification, RFID, tag configured to permit individual identification of the injection device, wherein the RFID tag includes a RFID chip and a RFID antenna, said supporting ring having an opening allowing mounting of the supporting ring around the distal tip such that the RFID tag is located between a proximal end of the adaptor and the distal shoulder of the barrel.

2. The injection device according to claim 1, wherein the supporting ring opening has an inner diameter that is lower than an outer diameter of the distal tip.

3. The injection device according to claim 1, wherein the supporting ring has an outer diameter that is equal to or lower than an outer diameter of the adaptor.

4. The injection device according to claim 1, wherein the supporting ring defines a distal abutment face configured to abut against the proximal end of the adaptor.

5. The injection device according to claim 1, wherein the supporting ring has a flat distal face and a flat opposite proximal face, and extends in a plane orthogonal to the longitudinal axis.

6. The injection device according to claim 1, wherein the supporting ring is secured to a proximal-most portion of the distal tip.

7. The injection device according to claim 1, wherein the supporting ring has a bevel extending around the opening, on a proximal face thereof.

8. The injection device according to claim 1, wherein the supporting ring has inward radial legs defining the opening and configured to secure the supporting ring to the distal tip.

9. The injection device according to claim 1, wherein the RFID antenna and the RFID chip are embedded, preferably overmolded, in the supporting ring.

10. The injection device according to claim 1, wherein the supporting ring is a Printed Circuit Board, PCB, the RFID antenna is directly printed on said PCB, preferably on a distal face thereof, and the RFID chip is welded to the RFID antenna.

11. The injection device according to claim 1, wherein the RFID tag is a Low Frequency Radio Frequency Identification, LF-RFID, tag, a High Frequency Radio Frequency Identification, HF-RFID, tag or, preferably, a Ultra High Frequency Radio Frequency Identification, UHF-RFID, tag.

12. A method for assembling the injection device according to claim 1, said method comprising the step of mounting the supporting ring onto the distal tip before mounting the adaptor onto said distal tip, such that the supporting ring including the RFID tag is arranged between a proximal end of the adaptor and a distal end of the barrel.

13. The method according to claim 12, wherein the method comprises the steps of:
(i) inserting the opening of the supporting ring onto the distal tip;
(ii) moving the supporting ring in the proximal direction relative to the distal tip, until an inner diameter (d) of the supporting ring; interferes with an outer diameter of the distal tip;
(iii) pushing the supporting ring in the proximal direction in order to move the supporting ring into a groove of the distal tip;
(iv) securing the adaptor to the distal tip.

14. The method according to claim 13, wherein the proximal movement of the supporting ring relative to the distal tip in step (ii) is accomplished by gravity.

15. The method according to claim 13, wherein step (iii) includes the supporting ring being pushed further in the proximal direction by the proximal end of the adaptor abutting against the supporting ring, until the adaptor reaches its final position relative to the distal tip.
